(19) United States
(12) Patent Application Publication
(10) Pub. No.: US 2024/0263021 A1
(43) Pub. Date: Aug. 8, 2024

(54) ANTIVIRAL AND ANTIMICROBIAL BARRIER COATINGS

(71) Applicants: GEORGIA TECH RESEARCH CORPORATION, Atlanta, GA (US); CHITOZAN HEALTH LLC, Jacksonville, FL (US)

(72) Inventors: James Carson Meredith, III, Marietta, GA (US); Austin VAN HORN, Jacksonville, FL (US)

(21) Appl. No.: 18/567,181

(22) PCT Filed: Jun. 9, 2022
(86) PCT No.: PCT/US2022/032843

§ 371 Date: Dec. 5, 2023

Related U.S. Application Data
(60) Provisional application No. 63/208,815, filed on Jun. 9, 2021.

(51) Int. Cl. C09D 5/14; A01N 59/16; A01N 59/20; A01P 1/00; C09D 105/08

(52) U.S. Cl. CPC C09D 5/14; A01N 59/16; A01N 59/20; A01P 1/00; C09D 105/08

(57) ABSTRACT

Disclosed are compositions comprising chitin nanofibers (ChNF) and silver chitosan (AgCh) and/or silver-copper chitosan (AgCuCh). Also disclosed are methods of coating various substrates with the disclosed compositions in order to impart antiviral and antimicrobial properties, as well as oxygen and water vapor barrier properties, to the substrate. Coated substrates made by the disclosed methods are also disclosed.

### CROSS REFERENCE TO RELATED APPLICATIONS

This application claims the benefit of priority to U.S. Provisional Application 63/208,815, filed Jun. 9, 2021, which is incorporated by reference herein in its entirety.

### ACKNOWLEDGEMENT OF GOVERNMENT FUNDING

This invention was made with government support under Grant No. DE-EE0008494 awarded by the U.S. Department of Energy. The government has certain rights in the invention.

### BACKGROUND

Acute shortages of PPE exist, and due to global supply chain disruption by COVID-19, shortages are expected to continue. The threat to medical personnel and other patients from viral or microbial infection from the surface of contaminated PPE has always been a problem, but has been made more challenging due to recent PPE shortages that require reuse of PPE. Treating PPE surfaces so that they can inactivate or kill viruses and other opportunistic pathogens can extend the lifetime of PPE considerably. Treated PPE can even be more effective than non-treated PPE, because it not only acts as a barrier to protect the wearer, but because it minimizes transmission of infections to other persons.

What are thus needed are antiviral and antimicrobial coatings for surfaces such as PPE that minimize transmission of a broad spectrum of secondary opportunistic pathogens, a significant contributor to mortality in hospital patients. Such coatings can be an attractive solution for surgeons and other infectious disease healthcare personnel, who can be protected during the doffing (removal) of contaminated PPE if the PPE surface has contact inhibition properties supplied by the coating. In a similar manner, outbreaks of food-borne infectious disease, particularly on the surfaces of produce, as well as infectious agents present in pharmaceutical preparations, indicate the value of applying an antiviral or antimicrobial (bacterial/fungal) to the surface of packaging materials for food, medicine or surgical instruments. The compositions and methods disclosed herein address these and other needs.

### SUMMARY

In accordance with the purposes of the disclosed materials and methods, as embodied and broadly described herein, the disclosed subject matter, in one aspect, relates to compounds, compositions and methods of making and using compounds and compositions. In specific aspects, the disclosed subject matter relates to compositions comprising chitin nanofibers (ChNF) and silver chitosan (AgCh) and/or silver-copper chitosan (AgCuCh). Also disclosed are methods of coating various substrates with the disclosed compositions in order to impart antiviral and antimicrobial properties, as well as oxygen and water vapor barrier properties, to the substrate. Coated substrates made by the disclosed methods are also disclosed.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURE

The accompanying FIGURE, which is incorporated in and constitutes a part of this specification, illustrates several aspects described below.

FIG. 1 is a group of photographs of 4 inch×4 inch films of cellulose acetate (CA) substrate, and its coatings with chitin nanofibers (CA-ChNF), and a 90/10 mixture of ChNF and silver-chitosan (AgCh) (CA-(ChNF+AgCh). Coatings were delivered by spray at ambient conditions from dilute aqueous suspension.

### DETAILED DESCRIPTION

The materials, compounds, compositions, and methods described herein may be understood more readily by reference to the following detailed description of specific aspects of the disclosed subject matter and the Examples and FIGURE included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a coating” includes mixtures of two or more such compositions, reference to “an additive” includes mixtures of two or more such additives, and the like.

“Optional” or “optionally” means that the subsequently described event or circumstance can or cannot occur, and that the description includes instances where the event or circumstance occurs and instances where it does not.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Unless stated otherwise, the term “about” means within 5% (e.g., within 2% or 1%) of the particular value modified by the term “about.”

As used herein, the term “composition” is intended to encompass a product comprising the specified ingredients in the specified amounts, as well as any product which results, directly or indirectly, from combination of the specified ingredients in the specified amounts.

References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5, and are present in such ratio regardless of whether additional components are contained in the mixture.

A weight percent (wt. %) of a component, unless specifically stated to the contrary, is based on the total weight of the formulation or composition in which the component is included.

Reference will now be made in detail to specific aspects of the disclosed materials, compounds, compositions, articles, and methods, examples of which are illustrated in the accompanying Examples and FIGURE.

### Compositions

Disclosed herein, in one aspect, are compositions comprising chitin nanofibers (ChNF) and silver chitosan (AgCh) and/or silver-copper chitosan (AgCuCh). The disclosed compositions can be used as antiviral and antimicrobial barrier coatings on various substrates. The disclosed compositions can be in the form of a suspension or solution, wherein the ChNF and AgCh and/or AgCuCh are present with a carrier liquid or solvent, respectively. The term “carrier” means substance that, when in combination with the disclosed compositions, aids or facilitates preparation, storage, administration, delivery, effectiveness, selectivity, or any other feature of the composition for its intended use or purpose. For example, a carrier can be selected to minimize any degradation of the disclosed compositions and to aid in its application to a substrate. Examples of suitable carrier liquids and solvents include water, acetic acid, methanol, ethanol, propanol, isopropanol, butanol, dimethyl ether, tetrahydrofuran, cyclohexanone, ethyl acetate, acetone, t-butylether, pentane, hexane, heptane, glycerol, dichloromethane, chloroform, dimethylformamide, dimethylsulfoxide, benzene, toluene, pyridine. The disclosed carrier liquids and solvents can also contain dilute acids.

### Chitosan and Chitin

Chitosan is derived from chitin, an abundantly available biopolymer with natural production being of the same order of magnitude as cellulose (Barikani, M., et al., Preparation and application of chitin and its derivatives: a review. Iranian Polymer J., 2014, 23(4):307-326). Chitosan is a linear polysaccharide and a homopolymer of D-glucosamine and produced by deacetylation of chitin (Hamed, I., et al., Industrial applications of crustacean by-products (chitin, chitosan, and chitooligosaccharides): A review. Trends Food Sci. Tech., 2016, 48:40-50). Due to the presence of secondary amino groups chitosan is soluble in dilute acids like acetic, formic, succinic etc. Chitin and chitosan can be thought of as the same material, but with different degrees of deacetylation and correspondingly different contents of secondary (—NH₂) amine group. Chitosan is chitin with a degree of acetylation of less than 50 mol %. Naturally-occurring chitin contains some fraction of chitosan distributed as a comonomer along the chain, and the process of extracting chitin from its parent source (such as crustacean exoskeleton) leads to some deacetylation, which can be carried out to an extreme degree to produce chitosan. While chitosan is soluble in dilute aqueous acids, chitin is insoluble and remains in solid fiber form. The solid fibers can be present as large aggregates or through processing, such as homogenization, can be reduced to nanocrystals or nanofibers. While the main examples provided herein is with chitin nanofibers, the disclosed compositions can use one or more other forms of chitin such as chitin nanocrystals, chitin nanowhiskers, as well chitosan nanofibers, nanowhiskers, and/or nanocrystals, which contain some quantity of secondary amine on their surfaces. Thus, it is expressly contemplated that the disclosed methods and compositions can replace chitin with chitosan in the matrix to which AgCh or AgCuCh is added.

Chitin and chitosan fibers are bacteriostatic and can be dissolved or suspended in water (with dilute acid added, such as acetic acid), and sprayed or otherwise coated into a liquid film that dries to form a solid coating. Chitin nanofibers (ChNF), nanocrystals (ChNCs) and nanowhiskers (ChNW) can dry to form transparent coatings because their dimensions are less than that of typical visible light wavelengths. Chitosan and larger chitin fibers can lead to hazy or opaque coatings.

The amount of ChNF, ChNC or ChNW (hereafter referred to as chitin fiber (CF)) in the disclosed compositions can be at least about 5 wt. %, e.g., at least about 10 wt. %, at least about 15 wt. %, at least about 20 wt. %, at least about 25 wt. %, at least about 30 wt. %, at least about 35 wt. %, at least about 40) wt. %, at least about 45 wt. %, at least about 50 wt. %, at least about 55 wt. %, at least about 60 wt. %, at least about 65 wt. %, at least about 70) wt. %, at least about 75 wt. %, at least about 80 wt. %, at least about 85 wt. %, at least about 90) wt. %, or at least about 95 wt. %. In other examples, the amount of ChF in the disclosed compositions can be less than about 95 wt. %, e.g., less than about 90 wt. %, less than about 85 wt. %, less than about 80 wt. %, less than about 75 wt. %, less than about 70 wt. %, less than about 65 wt. %, less than about 60 wt. %, less than about 55 wt. %, less than about 50) wt. %, less than about 45 wt. %, less than about 40 wt. %, less than about 35 wt. %, less than about 30 wt. %, less than about 25 wt. %, less than about 20 wt. %, less than about 15 wt. %, less than about 10 wt. %, or less than about 5 wt. %. In still other examples, the amount of ChF in the disclosed compositions can be about 5 wt. %, about 10 wt. %, about 15 wt. %, about 20 wt. %, about 25 wt. %, about 30 wt. %, about 35 wt. %, about 40 wt. %, about 45 wt. %, about 50 wt. %, about 55 wt. %, about 60 wt. %, about 65 wt. %, about 70 wt. %, about 75 wt. %, about 80 wt. %, about 85 wt. %, about 90 wt. %, or about 95 wt. %, where any of the stated values can form an upper or lower endpoint of a range. For example, the amount of ChF in the disclosed compositions can be from about 5 wt. % to about 95 wt. %, from about 25 wt. % to about 95 wt. %, from about 50 wt. % to about 95 wt. %, or from about 75 wt. % to about 95 wt. %.

### Silver and Silver Copper Chitosan

The disclosed compositions also comprise silver chitosan (AgCh) and/or silver-copper chitosan (AgCuCh). AgCh is an adduct formed by reaction between chitosan and silver nitrate (or other silver salts), that results in direct complexation between the amine moiety on chitosan and the silver atom. AgCuCh can be similarly formed with a combination of silver and copper salts and chitosan. AgCh and AgCuCh function to disrupt viral protein coats and bacterial cell walls. They are distinguished from other products in the silver antimicrobial class because they are not a nanoparticle-based formulation, but are a direct adduct of silver (and copper) salt with the amine group on chitosan monomer. Chitosan is GRAS and silver nitrate is on the FDA OTC monograph for consumer products. In some examples, the AgCh can be obtained by the processes disclosed in U.S. Pat. No. 7,700,131, which is incorporated by reference herein in its entirety for its teachings of methods of preparing silver containing chitin or chitosan adducts.

The amount of AgCh and/or AgCuCh in the disclosed compositions can be at least about 0.001 wt. %, e.g., at least about 0.01 wt. %, at least about 0.1 wt. %, at least about 1 wt. %, at least about 5 wt. %, at least about 10 wt. %, at least about 15 wt. %, at least about 20 wt. %, at least about 25 wt. %, at least about 30 wt. %, at least about 35 wt. %, at least about 40 wt. %, at least about 45 wt. %, at least about 50 wt. %, at least about 55 wt. %, at least about 60 wt. %, at least about 65 wt. %, at least about 70 wt. %, at least about 75 wt. %, at least about 80 wt. %, at least about 85 wt. %, at least about 90 wt. %, or at least about 95 wt. %. In other examples, the amount of AgCh and/or AgCuCh in the disclosed compositions can be less than about 95 wt. %, e.g., less than about 90 wt. %, less than about 85 wt. %, less than about 80 wt. %, less than about 75 wt. %, less than about 70 wt. %, less than about 65 wt. %, less than about 60 wt. %, less than about 55 wt. %, less than about 50 wt. %, less than about 45 wt. %, less than about 40 wt. %, less than about 35 wt. %, less than about 30 wt. %, less than about 25 wt. %, less than about 20 wt. %, less than about 15 wt. %, less than about 10 wt. %, less than about 5 wt. %, less than about 1 wt. %, less than 0.1 wt. %, less than about 0.01 wt. %, or less than about 0.001 wt. %. In still other examples, the amount of AgCh and/or AgCuCh in the disclosed compositions can be about 0.001 wt. %, about 0.01 wt. %., about 0.1 wt. %, about 1 wt. %, about 5 wt. %, about 10 wt. %, about 15 wt. %, about 20 wt. %, about 25 wt. %, about 30 wt. %, about 35 wt. %, about 40 wt. %, about 45 wt. %, about 50 wt. %, about 55 wt. %, about 60 wt. %, about 65 wt. %, about 70 wt. %, about 75 wt. %, about 80 wt. %, about 85 wt. %, about 90 wt. %, or about 95 wt. %, where any of the stated values can form an upper or lower endpoint of a range. For example, the amount of AgCh and/or AgCuCh in the disclosed compositions can be from about 0.001 wt. %, to about 10 wt. %, from about 0.001 wt. % to about 5 wt. %, from about 0.001 wt. % to about 1 wt. %, from about 0.01 wt. % to about 10 wt. %, from about 0.1 wt. % to about 5 wt. %, or from about 1 wt. % to about 10 wt. %.

### Additional Components

The disclosed compositions can also comprise cellulose, e.g., to tune the oxygen barrier properties of the coating. In specific examples, the cellulose is a cellulose nanocrystal (CNC) but may also include cellulose nanofibers (CNF).

The amount of cellulose in the disclosed compositions can be at least about 5 wt. %, e.g., at least about 10 wt. %, at least about 15 wt. %, at least about 20 wt. %, at least about 25 wt. %, at least about 30 wt. %, at least about 35 wt. %, at least about 40 wt. %, at least about 45 wt. %, at least about 50 wt. %, at least about 55 wt. %, at least about 60 wt. %, at least about 65 wt. %, at least about 70 wt. %, at least about 75 wt. %, at least about 80 wt. %, at least about 85 wt. %, at least about 90 wt. %, or at least about 95 wt. %. In other examples, the amount of cellulose in the disclosed compositions can be less than about 95 wt. %, e.g., less than about 90 wt. %, less than about 85 wt. %, less than about 80 wt. %, less than about 75 wt. %, less than about 70 wt. %, less than about 65 wt. %, less than about 60 wt. %, less than about 55 wt. %, less than about 50 wt. %, less than about 45 wt. %, less than about 40 wt. %, less than about 35 wt. %, less than about 30 wt. %, less than about 25 wt. %, less than about 20 wt. %, less than about 15 wt. %, less than about 10 wt. %, or less than about 5 wt. %. In still other examples, the amount of cellulose in the disclosed compositions can be about 5 wt. %, about 10 wt. %, about 15 wt. %, about 20 wt. %, about 25 wt. %, about 30 wt. %, about 35 wt. %, about 40 wt. %, about 45 wt. %, about 50 wt. %, about 55 wt. %, about 60 wt. %, about 65 wt. %, about 70 wt. %, about 75 wt. %, about 80 wt. %, about 85 wt. %, about 90 wt. %, or about 95 wt. %, where any of the stated values can form an upper or lower endpoint of a range. For example, the amount of cellulose in the disclosed compositions can be from about 5 wt. % to about 95 wt. %, from about 25 wt. % to about 75 wt. %, from about 30 wt. % to about 50 wt. %, or from about 50 wt. % to about 70 wt. %. In specific examples, the amount of cellulose in the disclosed compositions can be about 33 wt. % or about 50 wt. %.

In further examples, the disclosed compositions can also comprise binders, such as alginate, polyacrylic acid, polylactic acid, polyglycolic acid, hemicellulose, methyl cellulose, ethyl cellulose, hydropropylmethylcellulose, carboxy methylcellulose, hydroxypropyl cellulose, gellan gum, xanthan gum, pectin, starch, and any combination thereof.

The disclosed compositions can also comprise wetting agents, UV blockers, pigments, dyes, plasticizers, and viscosity modifiers.

### Coated Compositions

The disclosed compositions can be coated onto a variety of substrate surfaces to provide antiviral and antimicrobial properties to the surface. The coatings can also provide a barrier to oxygen and water vapor. In specific aspects, the coating is substantially clear. In many examples, the substrate can be plastic, such as a film, container, or a woven or nonwoven material made of plastic fibers. In specific examples the plastic can be a hydrophilic polymer. Examples of hydrophilic polymers include cellulose acetate, polyvinyl alcohol, polyethyleneimine, polyacrylic acid, polyethyleneglycol, polyvinylpyrrolidone, and copolymers thereof.

In other examples the plastic can be polyethylene terephthalate, polybutylene terephthalate polyester, phenol-formaldehyde, polyvinyl chloride, polyamide, polyurethane, nylon, polyolefins like polyethylene, polypropylene, polystyrene, poly butadiene, poly butadiene, or acrylonitrile-butadiene-styrene copolymer.

In still other examples, the substrate can be a textile, wool, wood, stone, or concrete. In still other examples, the substrate can be a paper-based materials including sheets and cardboard. In other examples, the substrate can be a molded item.

### Methods

The disclosed compositions can be coated onto a substrate by various methods. For example, a solution or suspension comprising ChF and AgCh and/or AgCuCh can be cast into a film on the substrate surface. Casting can be performed by pouring the solution/suspension onto a substrate, spinning the solution/suspension onto a substrate, spraying the solution/suspension onto a substrate, dipping a substrate into the solution/suspension, or by continuous coating including doctor-blade, knife-edge, slot-die, flexographic or gravure coating methods. The resulting film can be allowed to dry at ambient or elevated temperature.

The disclosed methods are distinguished from other methods where the substrate is contacted with chitosan and then a silver salt, followed by reducing the silver. Such methods typically do not result in clear coatings, nor do they have low oxygen and water vapor permeability. Instead, in the disclosed methods, silver chitosan (and/or silver-copper chitosan) is already prepared and combined with chitin nanofibers in a solution or suspension, which is then coated onto a substrate.

The resulting compositions can then be used to form films, wrappers, packing materials, packaging films, bandages, clothing, and other useful articles.

### Microbes

By coating a substrate with the disclosed compositions, the substrate can have antiviral and antimicrobial properties. Specific examples of bacteria that can be inhibited on a substrate surface by the disclosed compositions can include, but are not limited to, any bacterium found in the genera of Actinobacter, Actinomycetes, Bacilli, Bortedellen, Clostridia, Corynebacteria, Enterobacter, Enterococci, Escherichia, Helicobacter, Haemophilus, Klebsiella, Listeria, Mycobacteria, Neisseria, Shigella, Salmonella, tuberculosis bacteria, Yersinia, and Zymomonas. Specific examples include C. acetobutylicum, C. aerotolerans, C. beijerinckii, C. bifermentans, C. botulinum, C. butyricum, C. cadaveris, C. chauvoei, C. clostridioforme, C. colicanis, C. difficile, C. fallax, C. formicaceticum, C. histolyticum, C. innocuum, C. ljungdahlii, C. laramie, C. lavalense, C. novyi, C. oedematiens, C. paraputrificum, C. perfringens, C. phytofermentans, C. piliforme, C. ramosum, C. scatologenes, C. septicum, C. sordellii, C. sporogenes, C. sp, Q.D, C. tertium, C. tetani, C. tyrobutyricum, C. saccharoperbutylacetonicum, C. pasteurianum, C. thermocellum, C. cellulolyticum, C. saccharobutylicum, C. aurantibutyricum, C. tetanomorphum, Thermoanaerobacterium thermosaccharolyticum, Enterococcus faecium, E. coli, E. hirae, B. subtilis, B. stearothermophilus, B. licheniformis, B. polymyxa, B. licheniformis, B. amyloliquefaciens, Klebsiella pneumoniae, P. aeruginosa, P. polymyxa, S. aureus, Z. mobilis, and Acinetobacter baumannii.

Specific examples of fungi that can be inhibited on a substrate surface by the disclosed compositions can include, but are not limited to, fungi in the genus of Aspergillus, Candida, and Saccharomyces.

Specific examples of viruses that can be inhibited on a substrate surface by the disclosed compositions can include, but are not limited to, flaviviridae, picornaviridae, and coronaviridae, such as SARS and SARS-COV-2, and MERS.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

### Example 1: Antimicrobial and Antiviral Activity of AgCh and AgCuCh

AgCh inactivated an alphacoronavirus (feline coronavirus) by 99.98% (3.79 log reduction) in a quantitative suspension test (BS EN 14476:2013+A2:2019) and 99.69% (2.5 log reduction) in a textile coating test (ISO 18184:2019). Specifically, feline coronavirus was contacted for 1 min+/−5 s with a solution of AgCh at 37° C.

AgCh was also proven to be 99.98% effective against yeast and mold, passing the following tests: BS EN 16615, BS EN 13727, PAS 2424. It met the suspension test criteria for a medical surgical hand wash and is highly effective against a broad range of infectious bacteria

Also, 24-h testing show AgCh passed MRSA, E. coli, and P. aeruginosa log reduction standards. The product AgCh achieved a >5 log reduction against P. aeruginosa, S. aureus and E. hirae, when tested under clean conditions with a 5-minute contact time at a minimum concentration of 50%. BS EN 13727:2012+A2:2015

AgCuCh has been formulated for a different range of applications than AgCh, but has similar antimicrobial contact killing activity. In addition, the dispersability and spray-coating of AgCh and AgCuCh are similar.

### Example 2: Coating Clarity

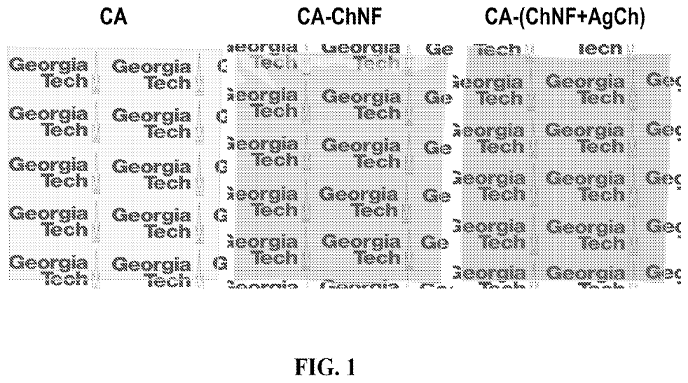

4×4 inch films of cellulose acetate (CA) were spray coated with chitin nanofibers (ChNF) or a 90/10 mixture of ChNF and AgCh in dilute aqueous solution. The coatings were clear as shown in FIG. 1. This example shows that ChNF and AgCh and/or AgCuCh can be blended in aqueous medium and sprayed onto plastic substrates to form transparent coatings.

### Example 3: Oxygen Permeability

Barrier properties of ChNF and cellulose nanocrystals (CNC) blended films are shown in Table 1. These materials can serve as excellent substrates for AgCh and AgCuCh additives to create oxygen-barrier, bacteriostatic coatings for packaging. The films containing ChNF have oxygen and water vapor barrier properties, in addition to bacteriostatic properties, which makes them effective barriers for protection of food and pharmaceutical products from both microbial pathogens as well as oxygen and water.

TABLE 1
Oxygen permeability properties of ChNF and CNC-containing
substrates for the AgCh coatings compared to commercial
oriented polyethylene terephthalate (PET) baseline.

| Material | OTR (for 25 μm thickness) |
|---|---|
| ChNF/CNC blend | 1 cm³ μm/(m² day kPa) |
| ChNF/CNC bilayer | 9 cm³ μm/(m² day kPa) |
| on cellulose acetate | |
| PET | 10 to 50 cm³ μm/(m² day kPa) |

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

1. A composition, comprising: chitin-nanofibers and silver chitosan and/or silver-copper chitosan.

2. The composition of claim 1, comprising chitosan-nanofibers and silver chitosan.

3. The composition of claim 1, comprising chitosan-nanofibers and silver-copper chitosan.

4. The composition of claim 1, wherein chitin nanofibers are at least about 5 wt. % of the composition.

5. The composition of claim 1, wherein silver chitosan and/or silver-copper chitosan are at least about 0.001 wt. % of the composition.

6. The composition of claim 1, further comprising cellulose.

7. The composition of claim 1, further comprising one or more of a binder, wetting agent, UV blocker, pigment, dye, plasticizer, or viscosity modifier.

8. A solution comprising the composition of claim 1 and a solvent.

9. A suspension comprising the composition of claim 1 and a carrier liquid.

10. A coated substrate, comprising: a substrate having a surface and the composition of claim 1.

11. The coated substrate of claim 10, wherein the substrate is a hydrophilic polymer.

12. The coated substrate of claim 11, wherein the hydrophilic polymer is cellulose acetate, polyvinyl alcohol, polyethyleneimine, polyacrylic acid, polyethyleneglycol, polyvinylpyrrolidone, and copolymers thereof.

13. The coated substrate of claim 10, wherein the substrate is polyethylene terephthalate, polybutylene terephthalate polyester, phenol-formaldehyde, polyvinyl chloride, polyamide, polyurethane, nylon, polyolefins like polyethylene, polypropylene, polystyrene, polybutadiene, polybutadiene, or acrylonitrile-butadiene-styrene copolymer.

14. The coated substrate of claim 10, wherein the substrate is a textile, wool, wood, stone, or concrete.

15. The coated substrate of claim 10, wherein the substrate is a paper-based material.

16. An article comprising a coated substrate of claim 10.

17. The article of claim 16, wherein the article is a glove, mask, gown, hat, foot cover, sheet, container, film, wrapper, or countertop.

18. A method of treating a substrate to provide antiviral and antimicrobial properties to the substrate, the method comprising: contacting a surface of the substrate with the composition of claim 1.

19. The method of claim 18, wherein contacting the surface is by pouring, spraying, or coating a solution or suspension comprising the composition onto the substrate, or by dipping the substrate into the solution or suspension comprising the composition.
